(19) United States
(12) Patent Application Publication
(10) Pub. No.: US 2015/0184248 A1
(43) Pub. Date: Jul. 2, 2015

(54) METHOD FOR DETECTING PANCREATIC CANCER AND DETECTION KIT

(71) Applicant: National Cancer Center, Chuo-ku, Tokyo (JP)

(72) Inventors: Naoto Tsuchiya, Tokyo (JP); Hiroko Ogata, Tokyo (JP); Takuji Okusaka, Tokyo (JP); Hitoshi Nakagama, Tokyo (JP)

(73) Assignee: National Cancer Center, Chuo-ku (JP)

(21) Appl. No.: 14/410,408

(22) PCT Filed: Jun. 26, 2013
(86) PCT No.: PCT/JP2013/067508

§ 371 Date: Dec. 22, 2014

(30) Foreign Application Priority Data
Jun. 27, 2012 (JP) 2012-144524

(51) Int. Cl. C12Q 1/68

(52) U.S. Cl. CPC C12Q 1/6886; C12Q 2600/178; C12Q 2600/158

(57) ABSTRACT

An object of the invention is to provide a simple and easy method for detecting pancreatic cancer having high sensitivity and specificity or risk of the pancreatic cancer.

The present invention provides a method of detecting pancreatic cancer including a step of measuring, in a sample derived from a subject, at least one miRNA selected from the following (i) and (ii):
(i) an miRNA having a sequence represented by any of SEQ ID NO: 1 to 120; and
(ii) an miRNA that hybridizes, under stringent conditions, with a nucleic acid having a sequence complementary to the miRNA (i) and having from 17 to 30 bases.

### TECHNICAL FIELD

The present invention relates to a method of detecting pancreatic cancer characterized by measuring a micro RNA specific to pancreatic cancer.

### BACKGROUND ART

A micro RNA (which may hereinafter be called “miRNA”) is one of intracellular small non-coding RNAs composed of about 22 bases and is a factor essential for controlling ontogenesis and cell differentiation. In many human diseases, miRNA dysfunction has been found. In particular, abnormal miRNA expression in cancer has been found in most types of cancer, suggesting that miRNA dysfunction is strongly associated with carcinogenesis. In recent years, it has been elucidated that miRNAs are released (secreted) outside cells while being enveloped in an exosome, which is an intracellular membrane vesicle. By the research of miRNAs, in addition to elucidation of the molecular mechanism of carcinogenesis, development of their application to therapeutic agents or diagnostic agents is expected.

The present inventors have already found an miRNA specific to colon cancer which will be a test marker of colon cancer (Patent Document 1).

One of the diseases which require an early detection strategy is pancreatic cancer. As pancreatic cancer tests, a variety of diagnostic imaging techniques and blood tests for detecting a tumor marker have conventionally been employed. Diagnostic imaging techniques need a large-scale equipment and an expensive apparatus and therefore, they are not widely prevalent. On the other hand, tumor markers which have hitherto been used do not have sufficient sensitivity and specificity and many of them do not increase until cancer progresses to some extent so that they are not suited for early detection.

Pancreatic cancer cannot be detected early because of having almost no early symptoms and it is refractory cancer which develops fast and has a poor prognosis. Since detection at an early stage enhances cure from pancreatic cancer, early detection is very important for reducing a mortality from pancreatic cancer.

### CITATION LIST

### Patent Document

Patent Document 1: International Publication No. 2011/040525

### DISCLOSURE OF THE INVENTION

### Problem to be Solved by the Invention

An object of the present invention is to provide a simple and easy method of detecting pancreatic cancer or a risk of the pancreatic cancer having high sensitivity and specificity.

### Means for Solving the Problem

The present inventors have proceeded with a study to overcome the above-mentioned problem and have identified miRNAs specific to pancreatic cancer by performing a profiling of exosomal miRNAs secreted from pancreatic cancer cell lines. In addition, the inventors have confirmed using clinical blood samples that the resulting profiles are greatly different from those of colon cancer. Further, the inventors have found that some of the miRNAs identified in the cell lines are detected at significantly high levels from blood samples of pancreatic cancer patients. As a result, the inventors have completed the present invention.

The present invention relates to:

[1] a method for detecting pancreatic cancer of a subject, including a step of measuring, in a sample derived from the subject, at least one miRNA selected from the following (i) and (ii):

(i) an miRNA having a sequence represented by any of SEQ ID NO: 1 to 120, and

(ii) an miRNA that hybridizes with a nucleic acid having a sequence complementary to the miRNA (i) under stringent conditions and having from 17 to 30 bases;

[2] the detection method as described above in [1], wherein the miRNA selected from (i) has a sequence represented by any of SEQ ID NO: 1 to 3, 5, 7, 12, 13, 19 to 21, 26, 27, 30, 32, 35, 36, 42, 50, 62 to 64, 70 to 73, 78, 80, 81, 82, and 85 to 120;

[3] the detection method as described above in either one of [1] or [2], wherein a concentration of the miRNA of the subject thus measured is statistically significantly higher than that of a healthy control, the subject is determined to suffer from pancreatic cancer;

[4] the detection method as described above in any of [1] to [3], wherein the sample derived from a subject is prepared by a method including a step of concentrating an exosome fraction from the plasma collected from the subject and a step of extracting an miRNA from the resulting exosome fraction;

[5] a kit for carrying out the detection method as described above in any of [1] to [4], including a solid-phase support having, fixed thereto, a DNA that hybridizes with the miRNA selected from (i) and (ii);

[6] a kit for carrying out the detection method as described above in any of [1] to [4], including a primer set necessary for amplifying an miRNA selected from (i) and (ii) or a nucleic acid complementary thereto by PCR assay;

[7] a method of detecting colon cancer of a subject, including a step of measuring the miRNA represented by SEQ ID NO: 47 in a sample derived from the subject;

[8] the method described above in [7], wherein the sample derived from the subject is prepared by a process including a step of concentrating an exosome fraction of the serum collected from the subject and a step of extracting an miRNA from the resulting exosome fraction;

[9] a kit for carrying out the detection method as described in [7] or [8], including a solid-phase support having, fixed thereto, a DNA that hybridizes with the miRNA represented by SEQ ID NO: 47; and

[10] a kit for carrying out the detection method as described above in [7] or [8], including a primer set necessary for amplifying the miRNA represented by SEQ ID NO: 47 or a nucleic acid complementary thereto by PCR assay.

### Effect of the Invention

According to the detection method of the present invention, the detection of pancreatic cancer or risk of the pancreatic cancer can be performed with high sensitivity and specificity, by a simple and easy method of measuring a predetermined miRNA in a sample of a subject.

### BRIEF DESCRIPTION OF THE DRAWINGS

FIG. 1 shows that an exosome fraction obtained from pancreatic cancer cell lines has a small molecular RNA in concentrated form.

FIG. 2 includes profiles of endogenous miRNA and exosomal miRNA derived from pancreatic cancer cell lines and immortalized pancreatic ductal epithelial cell lines, each profile obtained using a microarray analysis.

FIG. 3 is a typical example of comparison results of the profile of an exosomal miRNA derived from the plasma specimen of pancreatic cancer patients prepared using a microarray with analysis data of the plasma specimen of healthy controls.

FIG. 4 is a typical example of comparison results of the profile of an exosomal miRNA derived from the plasma specimen of pancreatic cancer patients prepared using a microarray with analysis data of the plasma specimen of healthy controls.

FIG. 5 shows the results of microarray profiling of exosomal miRNAs derived from the plasma specimen of colon cancer patients, pancreatic cancer patients, and healthy controls, respectively;

FIG. 6 shows the results of measuring expression of miR-23a-3p in exosomes derived from the plasma specimen of colon cancer patients and healthy controls, respectively.

FIG. 7 shows the results of measuring expression of miR-23a-3p in exosomes derived from plasma specimens of colon cancer patients before and after surgery, respectively.

### MODE FOR CARRYING OUT THE INVENTION

### Detection Method of Pancreatic Cancer

In one aspect, the method for detecting pancreatic cancer according to the present invention includes a step of measuring at least one miRNA selected from the following (i) and (ii):

(i) an miRNA having a sequence represented by any of SEQ ID NO: 1 to 120, and

(ii) an miRNA that hybridizes, under stringent conditions, with a nucleic acid having a sequence complementary to the miRNA (i) and having from 17 to 30 bases.

The “miRNA having a sequence represented by any of SEQ ID NO: 1 to 120” described above in (i) is, as will be described later in Examples, an miRNA secreted commonly by six pancreatic cancer cell lines or an miRNA detected at a significantly higher level in the plasma specimen derived from pancreatic cancer patients, compared with healthy controls.

Of these, the following 65 miRNAs were detected in the plasma specimen derived from pancreatic cancer patients at a significantly high level compared with healthy controls.

TABLE 1

| Name | SEQ ID NO: | Name | SEQ ID NO: | Name | SEQ ID NO: |
|---|---|---|---|---|---|
| hsa-lat-7a-5p | 1 | hsa-miR-141-3p | 93 | hsa-miR-493-5p | 105 |
| hsa-let-7f-5p | 5 | hsa-miR-144-5p | 94 | hsa-miR-494 | 106 |
| hsa-let-7i-5p | 7 | hsa-miR-1471 | 27 | hsa-miR-498 | 107 |
| hsa-miR-1268a | 19 | hsa-miR-150-3p | 95 | hsa-miR-513a-5p | 73 |
| hsa-miR-144-3p | 26 | hsa-miR-181c-3p | 96 | hsa-miR-518e-5p | 108 |
| hsa-miR-15a-5p | 30 | hsa-miR-184 | 97 | hsa-miR-525-5p | 109 |
| hsa-miR-16-5p | 32 | hsa-miR-1914-3p | 35 | hsa-miR-526b-5p | 110 |
| hsa-miR-22-3p | 42 | hsa-miR-193a-5p | 98 | hsa-miR-574-5p | 111 |
| hsa-miR-720 | 80 | hsa-miR-193b-5p | 36 | hsa-miR-584-5p | 112 |
| hsa-let-7b-5p | 2 | hsa-miR-202-3p | 99 | hsa-miR-623 | 113 |
| hsa-let-7c | 3 | hsa-miR-22-5p | 100 | hsa-miR-631 | 114 |
| hsa-miR-1182 | 86 | hsa-miR-25-3p | 50 | hsa-miR-652-3p | 115 |
| hsa-miR-1183 | 87 | hsa-miR-32-3p | 101 | hsa-miR-662 | 116 |
| hsa-miR-1207-5p | 12 | hsa-miR-320a | 102 | hsa-miR-671-5p | 78 |
| hsa-miR-1208 | 88 | hsa-miR-320b | 62 | hsa-miR-765 | 81 |
| hsa-miR-122-5p | 89 | hsa-miR-320c | 63 | hsa-miR-874 | 82 |
| hsa-miR-1224-5p | 13 | hsa-miR-320d | 64 | hsa-miR-885-3p | 117 |
| hsa-miR-1226-5p | 90 | hsa-miR-324-3p | 103 | hsa-miR-92b-5p | 118 |
| hsa-miR-124-3p | 91 | hsa-miR-371a-5p | 104 | hsa-miR-934 | 119 |
| hsa-miR-1275 | 20 | hsa-miR-451a | 70 | hsa-miR-936 | 120 |
| hsa-miR-1287 | 92 | hsa-miR-483-5p | 71 | hsa-miR-939 | 85 |
| hsa-miR-1290 | 21 | hsa-miR-486-5p | 72 | | |

The term “miRNA that hybridizes with a nucleic acid having a sequence complementary to the miRNA (i) under stringent conditions and having from 17 to 30 bases” means a mutant having high sequence identity with the miRNA (i). The term “high sequence identity” means that the mutant has 80% or more, 85% or more, 90% or more, 95% or more, or 98% or more sequence identity at a nucleic acid level.

The miRNA (ii) includes, for example, an miRNA (i) having any of the above-mentioned sequences wherein one or two bases are deleted, added, or substituted. The site to be deleted, added, or substituted may be either the 5′ end or the 3′ end of the miRNA (i), or may be a portion other than the end. The miRNA (ii) may have, for example, from 17 to 25 bases, or may have preferably the same number of bases as the miRNA (i) corresponding thereto.

The term “stringent conditions” as used herein means, for example, washing conditions at 65° C. in a 6×SSC solution (1×SSC: 0.15M NaCl, 15 mM sodium citrate) containing a 5% Denhardt's Solution (containing 0.1% Ficoll (Pharmacia), 0.1% polyvinylpyrrolidone, and 0.1% bovine serum albumin), 0.5% SDS, and 100 μg/ml of a salmon sperm DNA. The stringency can be controlled by a salt concentration (ionic strength), temperature, or the like. Under conditions with higher stringency, that is, under conditions with a lower salt concentration and a higher temperature, background due to nonspecific hybridization is removed by washing and only a nucleic acid that has specifically hybridized remains. Those skilled in the art can select stringent conditions as needed by regulating the temperature or salt concentration.

In the method for detecting pancreatic cancer in the present invention, it is only necessary to measure at least one miRNA selected from (i) and (ii). In other words, pancreatic cancer or risk of pancreatic cancer may be determined measuring one miRNA or two or more miRNAs.

When two or more miRNAs are used in combination, the number of them to be combined is not limited. For example, it is preferred to use two or more miRNAs selected from 65 miRNAs shown in Table 1 in combination.

The term “a step of measuring miRNA” as used herein may mean a step of accurately measuring the concentration of miRNA in a sample, a step of measuring the concentration not accurately insofar as the concentration in the sample can be confirmed to be significantly higher than the concentration level of a healthy control, or a step of only detecting whether the miRNA is contained in the sample or not.

In particular, with regard to miRNAs hardly detectable from a sample of healthy controls, it is only necessary to find whether the sample from subjects contains these miRNAs or not.

The step of measuring miRNA may be performed by determining the concentration of each miRNA, or by comparing, as a whole, an expression profile (expression pattern) of a plurality of miRNAs contained in a sample and an expression profile of these miRNAs in a healthy control and then, evaluating their similarity.

A measuring method of the miRNA is not particularly limited and any methods capable of detecting/measuring RNAs having a specific sequence in a sample can be used. Examples of such a method include northern hybridization assay, RNase protection assay, quantitative PCR assay such as RT-PCR assay or real time PCR assay, and assay using a DNA microarray.

In northern hybridization assay, after extracted miRNAs are developed on a gel by using electrophoresis such as agarose gel electrophoresis and transferred onto a membrane such as nitrocellulose membrane or nylon membrane, a target miRNA is detected by using a probe that hybridizes with the miRNA (target miRNA) selected from (i) and (ii). For labeling of the probe, radioisotopes such as ³²P, digoxigenin (DIG) with its antibody, or the like can be used.

The RNase protection assay is a method of hybridizing an RNA probe labeled with a radioisotope or DIG with a target miRNA, digesting, with an RNase specific to a single strand, a region of the probe that has not hybridized with the target miRNA, and detecting a portion of the probe protected by hybridization through electrophoresis or the like.

The RT-PCR assay is a method of obtaining a cDNA from the miRNA in a sample by using a reverse transcriptase, carrying out PCR with the resulting cDNA as a template while using an appropriate primer, and analyzing the amplified product through electrophoresis or the like to determine the RNA amount in the sample.

The real time PCR assay is a technique of using fluorescence for detecting a PCR amplified product. It includes an intercalation method using a fluorescent label to be inserted specifically to a double-stranded nucleic acid typified by SYBR Green and a technique using a fluorescent-labeled and sequence-specific probe typified by a TaqMan probe. Also when the real time PCR assay is used, a cDNA obtained from the miRNA in a sample by using a reverse transcriptase can also be used as a template.

The DNA microarray method is a technique of bringing DNA probes that hybridize with the entire or a portion of the target miRNAs and are immobilized on a solid-phase support into contact with a sample and detecting the target miRNA that has hybridized with the probe. It is suited for comprehensive profiling of the miRNA expression in the sample. The target miRNA can also be detected by designing so that a portion of the miRNA hybridizes with a probe immobilized to a solid-phase support and a remaining portion of the miRNA hybridizes with a labeled second DNA probe.

In one aspect of the detection method of the present invention, a sample derived from the subject is not particularly limited, but it is preferably a blood sample, particularly, serum or plasma.

Prior to the step of measuring miRNA, a step of concentrating an exosome fraction of the serum or plasma may be performed. It may be concentrated not only by ultracentrifugation but also by using a membrane, an antibody, or a reagent capable of specifically concentrating an exosome. Further, a step of extracting an miRNA from the exosome fraction may be performed. These steps may be carried out in a conventional manner by those skilled in the art.

The term “pancreatic cancer” as used herein has a typical meaning and it embraces pancreatic cancer in any state, not limited by pathological classification, form, invasion depth, staging showing progression, or the like.

The term “detect” as used herein means analyzing a sample collected from a subject in order to get data necessary for diagnosis. The detection method of the present invention can be performed, for example, by a test company.

The term “detect” in the present invention totally includes, in addition to finding whether the subject suffers from pancreatic cancer or not, analyzing a risk whether the subject is likely to suffer from pancreatic cancer; screening the possibility of pancreatic cancer even when no symptoms of it have yet appeared; and studying the tendency of cancer such as its progression, prognosis, therapeutic effect, likelihood of recurrence, and the like after the subject is found to suffer from pancreatic cancer.

The detection method according to the present invention may be used in combination with a test method using a tumor marker or diagnostic imaging which has been used conventionally for the test of pancreatic cancer.

The term “statistically significant” as used herein means, for example, that a risk ratio (significance level) of a value obtained by measurement is smaller than 0.05, 0.01, or 0.001. The term “statistically significantly higher” referring to the measured value means that when a quantitative difference of an miRNA obtained from a subject and that obtained from a healthy control is statistically processed, there is a significant difference between them and at the same time, the amount of the miRNA of the subject is higher than that of the healthy control. For verification of statistical processing, a known statistical test capable of determining presence or absence of significance may be used as needed and it is not particularly limited. For example, Student's t test and multiple comparison test can be used.

(Detection Kit)

The present invention also embraces a kit for carrying out the above-mentioned detection method according to the present invention.

In one aspect, the detection kit according to the present invention includes a solid-phase support having, immobilized thereon, DNAs that hybridize with miRNAs selected from (i) and (ii). The solid-phase support is not particularly limited insofar as it is a support capable of immobilizing thereon a DNA. Examples include microtiter plates made of glass, a metal, or a resin, substrates, beads, nitrocellulose membranes, nylon membranes, and PVDF membranes. DNAs can be immobilized to such a solid-phase support by a known method.

The detection kit may be equipped further with, for example, reaction/detection reagents, buffers, instruction manual, and the like.

The one aspect of the detection kit according to the present invention includes a primer set necessary for amplifying the miRNA selected from (i) and (ii) or a nucleic acid complementary thereto by PCR assay. As described above, in the method of measuring the amount of a target miRNA in a sample by PCR assay, it is the common practice to prepare a cDNA by using a reverse transcriptase. Therefore, the detection kit according to the present invention may be equipped with a primer set capable of amplifying the target miRNA or a DNA complementary thereto. Such a primer set can be designed as needed by those skilled in the art based on the sequence of the target miRNA.

Such a kit may be equipped further with, for example, reverse transcriptase, reaction/detection reagent, buffer, instruction manual, and the like.

(Diagnostic Method of Pancreatic Cancer)

The present invention also includes a method of diagnosing pancreatic cancer including a step of measuring at least one miRNA selected from the above-mentioned (i) and (ii) in a blood sample derived from a subject.

The term “diagnosing” as used herein means that those engaged in medical practice determine whether the subject suffers from a specific disease or not based on the detection results and the like.

Terms used in the method of diagnosing pancreatic cancer according to the present invention which are also used in the detection method according to the present invention have the same meanings as those used in the detection method and a description on them is omitted here.

(Method of Detecting Colon Cancer)

The present application also embraces a method of detecting colon cancer including a step of measuring the miRNA having a sequence represented by SEQ ID NO: 47 in a blood sample derived from a subject or an miRNA that hybridizes with a nucleic acid having a sequence complementary to the miRNA under stringent conditions and having from 17 to 30 bases. The miRNA represented by SEQ ID NO: 47 has been named hsa-miR-23a-3p.

Terms used in the method of detecting colon cancer which are also used in the detection method of pancreatic cancer have the same meanings as those used in the detection method.

As the sample, blood samples such as serum and plasma are preferred.

As will be described later in Examples, this miRNA is hardly contained in the sample of a healthy control, but is frequently contained in a high concentration in the sample of a colon cancer patient even if at an early stage. Compared with the concentration before operation of colon cancer, the concentration shows a significant decrease after operation so that it is useful also for postoperative follow-up.

The hsa-miR-23a-3p can be used either singly as a colon cancer marker or in combination, for example, with at least one of the miRNAs disclosed in Patent Document 1.

The present invention includes a colon cancer detection kit containing a solid-phase support having, immobilized thereon, a DNA that hybridizes with the miRNA having a sequence represented by SEQ ID NO: 47. No particular limitation is imposed on the solid-phase support insofar as it permits immobilization of a DNA thereon and examples include microtiter plates made of glass, a metal, a resin, or the like, substrates, beads, nitrocellulose membranes, nylon membranes, and PVDF membranes. The DNA can be immobilized onto these solid-phase supports in a known manner. The kit may be equipped further with, for example, reaction/detection reagents, buffers, instruction manual, and the like.

The present invention also includes a colon cancer detection kit including a primer set necessary for amplifying the miRNA having a sequence represented by SEQ ID NO: 47 or a nucleic acid complementary thereto by PCR assay. Such a primer set can be designed as needed by those skilled in the art based on the sequence of the target miRNA. Such a kit may be equipped further with, for example, reverse transcriptase, reaction/detection reagents, buffers, and instruction manual.

The present invention also includes a method of diagnosing colon cancer, including a step of measuring, in a sample derived from a subject, the miRNA having a sequence represented by SEQ ID NO: 47 or an miRNA that hybridizes with a nucleic acid having a sequence complementary to the miRNA under stringent conditions.

All the disclosure of the patent documents and non-patent documents cited herein is incorporated herein in its entirety.

### EXAMPLES

The present invention will hereinafter be described specifically based on Examples, but the present invention is not limited to them. The present invention can be changed into various aspects without departing from the spirit of the present invention and such changes are also embraced in the scope of the present invention.

1. Isolation of Exosome and Preparation of Profile of Exosomal miRNA

Six pancreatic cancer cell lines (BxPC3, CAPAN1, HPAFII, Hs766T, PANC1, and PSN1) and two immortalized pancreatic ductal epithelial cell lines (HPDE4 and HPDE6) were cultured in a 10% FBS-containing medium in a 100-mm petri dish. Forty eight hours after the medium was replaced by a new one, an exosome fraction in each of the supernatants was concentrated by gradual ultracentrifugation (16,500×g for 20 min, 120,000×g for 70 min), RNAs were prepared by column purification, and it was used as a template of microarray analysis. Centrifugation at 16,500×g for 20 minutes was followed by filtration through a 0.2-1 μm nylon membrane. The filtrate thus obtained was used as a sample for subsequent centrifugation.

Profiling of the exosomal miRNA was performed using an miRNA microarray of Agilent Technologies according to its instruction manual and data were analyzed using GeneSpring X.

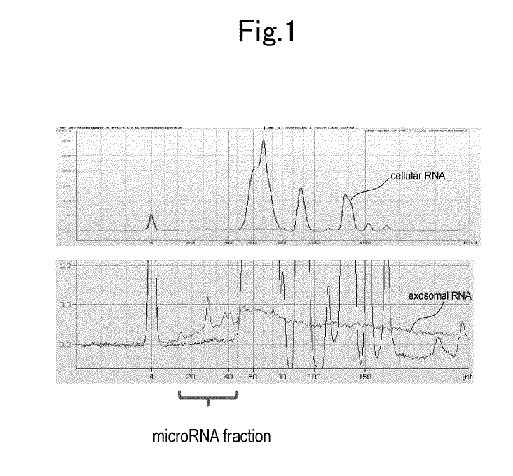

As shown in FIG. 1, an RNA as short as from about 20 to 30 bases was concentrated in the exosome fraction obtained by concentration through ultracentrifugation.

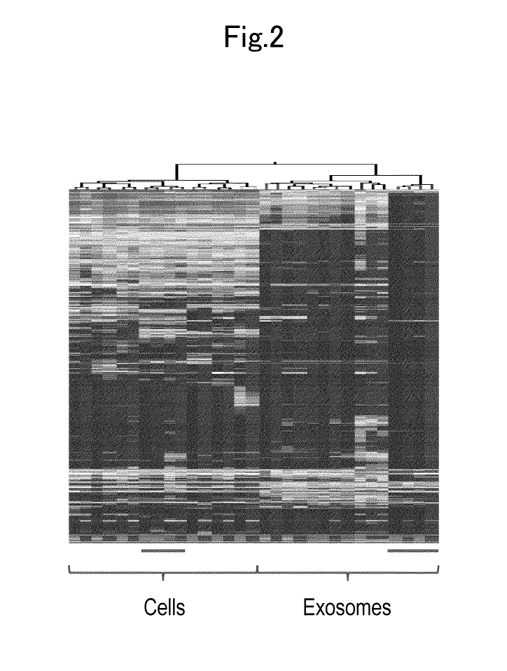

Results of microarray analysis performed using the resulting RNA sample are shown in FIG. 2.

As shown in FIG. 2, the profile of the exosomal miRNA secreted by pancreatic cancer cells is largely different from the expression profile of endogenous miRNA, revealing that the miRNA secreted by pancreatic cancer cells has selectivity.

Interestingly, the cancer cell lines cannot be distinguished from the immortalized cells (underlined portion in this drawing) in the endogenous expression profile (left panel), but they can be distinguished from each other in the exosomal miRNA profile.

This result suggests the possibility that a change in the exosomal miRNA profile is induced by malignant transformation (cancerization) of cells.

Then, the miRNAs secreted commonly by the six pancreatic cancer cells was selected. Microarray data were normalized by each of the number of cells, miR-923 (ribosome RNA), and total RNA amount. As shown in the following table, 85 miRNAs expressed in the pancreatic cancer cell lines were identified.

TABLE 2

| panc cancer cells exosomes | | | | | | | |
|---|---|---|---|---|---|---|---|
| Correction | Number of | | RNA | Number of | | RNA | |
| miR (85miR) having a signifi- | cells (×10\hat{ }7) | miR-923 | amount(ng) | cells (×10\hat{ }7) | miR-923 | amount(ng) | |
| cant difference (p < 0.05) | 79 | 47 | 35 | FC(6 Cancer cells/2Normal cells) | | | SEQ ID NO: |
| hsa-let-7a-5p | ◯ | ◯ | ◯ | 25.6 | 5 | 8.64 | 1 |
| hsa-let-7b-5p | ◯ | ◯ | | 12.1 | 2.4 | | 2 |
| hsa-let-7c | ◯ | | | 15.4 | | | 3 |
| hsa-let-7d-5p | ◯ | | | 11.2 | | | 4 |
| hsa-let-7f-5p | ◯ | ◯ | ◯ | 25.6 | 4.8 | 7.9 | 5 |
| hsa-let-7g-5p | ◯ | | | 15.5 | | | 6 |
| hsa-let-7i-5p | ◯ | | ◯ | 35 | | 5.6 | 7 |
| hsa-miR-103a-3p | ◯ | | | 15.6 | | | 8 |
| hsa-miR-106b-5p | ◯ | ◯ | ◯ | 35.4 | 4.1 | 7.5 | 9 |
| hsa-miR-107 | ◯ | ◯ | ◯ | 30.6 | 3.1 | 6 | 10 |
| hsa-miR-1181 | ◯ | | | 11 | | | 11 |
| hsa-miR-1207-5p | ◯ | | | 5.9 | | | 12 |
| hsa-miR-1224-5p | ◯ | | | 12.1 | | | 13 |
| hsa-miR-1246 | ◯ | ◯ | ◯ | 13.7 | 2.5 | 2.6 | 14 |
| hsa-miR-125a-3p | | ◯ | ◯ | | 4.3 | 9.6 | 15 |
| hsa-miR-125b-2-3p | ◯ | | | 7.6 | | | 16 |
| hsa-miR-126-3p | ◯ | ◯ | | 20.7 | 2 | | 17 |
| hsa-miR-1260a | ◯ | | | 3.1 | | | 18 |
| hsa-miR-1268a | ◯ | | | 8 | | | 19 |
| hsa-miR-1275 | ◯ | ◯ | ◯ | 22.7 | 3.6 | 3 | 20 |
| hsa-miR-1290 | ◯ | ◯ | ◯ | 20 | 5.9 | 4.4 | 21 |
| hsa-miR-1305 | ◯ | ◯ | ◯ | 28.6 | 2.9 | 5.7 | 22 |
| hsa-miR-134 | | ◯ | | | 2.3 | | 23 |
| hsa-miR-135a-3p | ◯ | | | 14.8 | | | 24 |
| hsa-miR-142-3p | ◯ | ◯ | ◯ | 26.2 | 2.9 | 6.3 | 25 |
| hsa-miR-144-3p | ◯ | ◯ | ◯ | 51.2 | 6.8 | 13.5 | 26 |
| hsa-miR-1471 | ◯ | | | 12.5 | | | 27 |
| hsa-miR-150-5p | ◯ | | | 8.9 | | | 28 |
| hsa-miR-151a-5p | ◯ | | | 6.4 | | | 29 |
| hsa-miR-15a-5p | ◯ | ◯ | | 22.1 | 2.3 | | 30 |
| hsa-miR-15b-5p | ◯ | ◯ | ◯ | 39.8 | 4.1 | 8.2 | 31 |
| hsa-miR-16-5p | ◯ | ◯ | ◯ | 88.8 | 9.4 | 18.8 | 32 |
| hsa-miR-17-5p | ◯ | ◯ | | 19.1 | 2.3 | | 33 |
| hsa-miR-181a-5p | ◯ | | | 7.1 | | | 34 |
| hsa-miR-1914-3p | ◯ | | | 15.2 | | | 35 |
| hsa-miR-193b-5p | ◯ | | | 16.1 | | | 36 |
| hsa-miR-19a-3p | ◯ | ◯ | | 20 | 2.3 | | 37 |
| hsa-miR-19b-3p | ◯ | ◯ | ◯ | 31.7 | 6.3 | 11.8 | 38 |
| hsa-miR-20a-5p | ◯ | ◯ | ◯ | 21.8 | 4.6 | 8.4 | 39 |
| hsa-miR-20b-5p | ◯ | | | 9.6 | | | 40 |

TABLE 3

| | | | | | | | |
|---|---|---|---|---|---|---|---|
| hsa-miR-21-5p | ◯ | ◯ | ◯ | 35.4 | 8.8 | 6.1 | 41 |
| hsa-miR-22-3p | ◯ | ◯ | ◯ | 54.8 | 5.6 | 11.2 | 42 |
| hsa-miR-221-3p | ◯ | ◯ | ◯ | 42.1 | 3.4 | 7.1 | 43 |
| hsa-miR-222-3p | ◯ | | | 9.6 | | | 44 |
| hsa-miR-223-3p | ◯ | ◯ | ◯ | 53.8 | 5.5 | 12.6 | 45 |
| hsa-miR-224-5p | ◯ | | | 8.7 | | | 46 |
| hsa-miR-23a-3p | ◯ | ◯ | ◯ | 11.1 | 3.1 | 2.4 | 47 |
| hsa-miR-23b-3p | ◯ | ◯ | ◯ | 24.7 | 3.2 | 5.8 | 48 |
| hsa-miR-24-3p | ◯ | ◯ | ◯ | 21.3 | 5.1 | 4.3 | 49 |
| hsa-miR-25-3p | ◯ | ◯ | ◯ | 64.3 | 6.4 | 13.3 | 50 |
| hsa-miR-26a-5p | ◯ | ◯ | | 14.9 | 1.7 | | 51 |
| hsa-miR-25b-5p | ◯ | | | 15 | | | 52 |
| hsa-miR-27a-3p | ◯ | ◯ | ◯ | 20.7 | 3.6 | 6.2 | 53 |
| hsa-miR-27b-3p | ◯ | ◯ | | 19.5 | 2.4 | | 54 |
| hsa-miR-29a-3p | ◯ | ◯ | ◯ | 55.1 | 6.2 | 12.9 | 55 |
| hsa-miR-29c-3p | ◯ | | | 16.2 | | | 56 |
| hsa-miR-30a-5p | ◯ | | | 8.2 | | | 57 |
| hsa-miR-30b-5p | ◯ | | | 6.4 | | | 58 |
| hsa-miR-30c-5p | ◯ | | | 5.1 | | | 59 |
| hsa-miR-30d-5p | ◯ | | | 21.8 | | | 60 |
| hsa-miR-30e-5p | ◯ | | | 6.7 | | | 61 |
| hsa-miR-320b | ◯ | ◯ | | 15.1 | 1.6 | | 62 |
| hsa-miR-320c | | ◯ | ◯ | | 3.9 | 5 | 63 |
| hsa-miR-320d | ◯ | | | 2.6 | | | 64 |
| hsa-miR-331-3p | ◯ | | | 6.8 | | | 65 |
| hsa-miR-345-5p | ◯ | ◯ | ◯ | 27.8 | 2.2 | 5.4 | 66 |
| hsa-miR-365a-3p | ◯ | ◯ | | 12.1 | 1.4 | | 67 |
| hsa-miR-423-5p | | ◯ | | | 1.7 | | 68 |
| hsa-miR-425-5p | ◯ | | | 11.9 | | | 69 |
| hsa-miR-451a | ◯ | ◯ | ◯ | 3799.9 | 421.9 | 909.1 | 70 |
| hsa-miR-483-5p | | ◯ | | | 2.9 | | 71 |
| hsa-miR-486-5p | ◯ | ◯ | ◯ | 43 | 5.4 | 10.7 | 72 |
| hsa-miR-513a-5p | ◯ | ◯ | ◯ | 171.3 | 4.3 | 5 | 73 |
| hsa-miR-601 | ◯ | | | 6.7 | | | 74 |
| hsa-miR-610 | ◯ | | | 10.8 | | | 75 |
| hsa-miR-630 | ◯ | | | 16.6 | | | 76 |
| hsa-miR-654-5p | | ◯ | ◯ | | 7.7 | 15.1 | 77 |
| hsa-miR-671-5p | ◯ | | | 1.8 | | | 78 |
| hsa-miR-708-5p | ◯ | | | 7.2 | | | 79 |
| hsa-miR-720 | ◯ | | | 3.7 | | | 80 |
| hsa-miR-765 | | ◯ | ◯ | | 9.3 | 27.2 | 81 |
| hsa-miR-874 | ◯ | ◯ | | 23.1 | 2.3 | | 82 |
| hsa-miR-92a-3p | ◯ | ◯ | ◯ | 25 | 8.2 | 15.3 | 83 |
| hsa-miR-93-5p | ◯ | ◯ | ◯ | 44.6 | 5.1 | 9.9 | 84 |
| hsa-miR-939 | ◯ | | | 5.1 | | | 85 |

2. Measurement of Exosomal miRNA Using Plasma of Pancreatic Cancer Patients (1)

Next, profiling of an exosomal miRNA was performed using plasma specimens (n=12) derived from pancreatic cancer patients and it was studied whether miRNA candidates specific to pancreatic cancer can be detected in clinical specimens or not.

First, the plasma specimen (from 0.75 to 1 mL) of each of pancreatic cancer patients was diluted to 10 times with PBS and then, the exosome fraction was concentrated by ultracentrifugation.

More specifically, array data of the plasma specimens of 10 healthy controls and 12 pancreatic cancer patients before chemotherapy were calculated as a signal intensity. After correction with the plasma volume, thus calculated value was designated as a normalized signal intensity (AU).

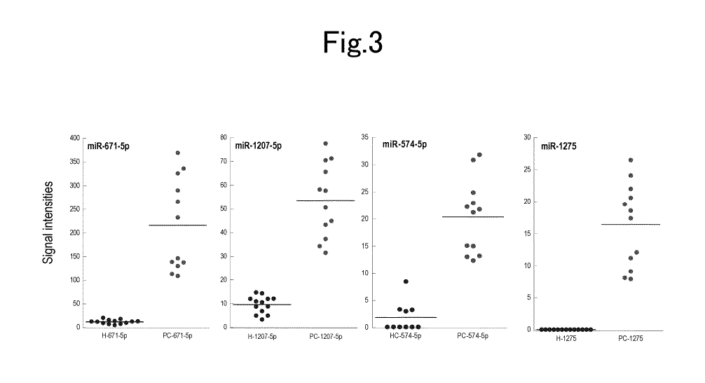

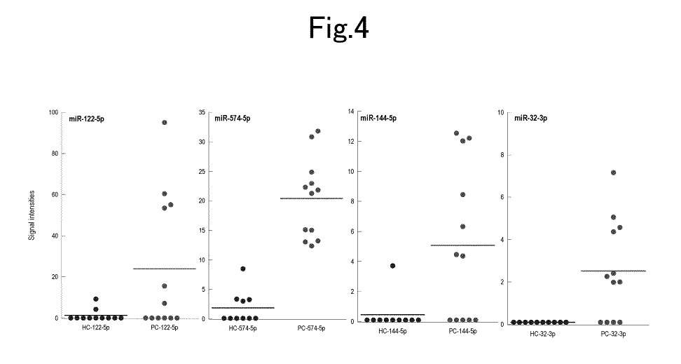

For analysis, T test was performed using a signal value of each of the miRNAs shown in terms of % supposing that the sum of the signal intensities of all the miRNAs detected was 100%. Those having a p value not more than 0.05 was selected as an miRNA specific to the plasma of pancreatic cancer patients. The results are shown below. Typical measurement examples are shown in FIGS. 3 and 4.

TABLE 4
Significantly higher microRNAs in plasma exosomes of
pancreatic cancer patients than healthy controls

| miR (56 miR) having | | | | | | | |
|---|---|---|---|---|---|---|---|
| significant | | HC (n = 10) | | PC (n = 12) | | Fold Change | SEQ |
| difference (p < 0.05) | Sequence | mean (%) | SD | mean (%) | SD | (PC/HC)) | ID NO: |
| hsa-let-7b-5p | ugagguaguagguugugugguu | 0.184 | 0.127 | 0.306 | 0.155 | 1.7 | 2 |
| hsa-let-7c | ugagguaguagguuguaugguu | 0.001 | 0.000 | 0.085 | 0.066 | 85.4 | 3 |
| hsa-miR-1182 | gagggucuugggagggaugugac | 0.001 | 0.000 | 0.175 | 0.191 | 175.3 | 86 |
| hsa-miR-1183 | cacuguaggugauggugagagugggca | 0.001 | 0.000 | 0.109 | 0.121 | 109.0 | 87 |
| hsa-miR-1207-5p | uggcagggaggcugggagggg | 0.458 | 0.141 | 0.878 | 0.295 | 1.9 | 12 |
| hsa-miR-1208 | ucacuguucagacaggcgga | 0.001 | 0.000 | 0.125 | 0.137 | 125.4 | 88 |
| hsa-miR-122-5p | uggagugugacaaugguguuug | 0.042 | 0.090 | 0.399 | 0.517 | 9.5 | 89 |
| hsa-miR-1224-5p | gugaggacucgggaggugg | 0.001 | 0.000 | 0.203 | 0.202 | 203.0 | 13 |
| hsa-miR-1226-5p | gugagggcaugcaggccuggaugggg | 0.001 | 0.000 | 0.113 | 0.125 | 113.3 | 90 |
| hsa-miR-124-3p | uaaggcacgcggugaaugcc | 0.001 | 0.000 | 0.096 | 0.073 | 95.9 | 91 |
| hsa-miR-1275 | gugggggagaggcuguc | 0.001 | 0.000 | 0.270 | 0.118 | 269.8 | 20 |
| hsa-miR-1287 | ugcuggaucagugguucgaguc | 0.335 | 0.177 | 0.762 | 0.492 | 2.3 | 92 |
| hsa-miR-1290 | uggauuuuuggaucaggga | 0.021 | 0.237 | 0.406 | 0.199 | 2.0 | 21 |
| hsa-miR-141-3p | uaacacugucugguaaagaugg | 0.001 | 0.000 | 0.108 | 0.122 | 108.1 | 93 |
| hsa-miR-144-5p | ggauaucaucauauacuguaag | 0.009 | 0.029 | 0.089 | 0.082 | 9.8 | 94 |
| hsa-miR-1471 | gcccgcguguggagccaggugu | 0.001 | 0.000 | 0.117 | 0.130 | 116.7 | 27 |
| hsa-miR-150-3p | cugguacaggccugggggacag | 0.073 | 0.230 | 0.417 | 0.322 | 5.7 | 95 |
| hsa-miR-181c-3p | aaccaucgaccguugaguggac | 0.001 | 0.000 | 0.027 | 0.031 | 27.1 | 96 |
| hsa-miR-184 | uggacggagaacugauaagggu | 0.001 | 0.000 | 0.157 | 0.173 | 156.7 | 97 |
| hsa-miR-1914-3p | ggaggggucccgcacugggagg | 0.001 | 0.000 | 0.138 | 0.152 | 137.7 | 35 |
| hsa-miR-193a-5p | ugggucuuugcgggcgagauga | 0.001 | 0.000 | 0.602 | 0.380 | 602.4 | 98 |
| hsa-miR-193b-5p | cgggguuuugagggcgagauga | 0.001 | 0.000 | 0.335 | 0.300 | 335.0 | 36 |
| hsa-miR-202-3p | agagguauagggcaugggaa | 0.001 | 0.000 | 0.127 | 0.137 | 126.5 | 99 |
| hsa-miR-22-5p | aguucuucaguggcaagcuuua | 0.001 | 0.000 | 0.312 | 0.328 | 311.6 | 100 |
| hsa-miR-25-3p | cauugcacuugucucggucuga | 0.039 | 0.064 | 0.106 | 0.075 | 2.7 | 50 |
| hsa-miR-32-3p | caauuuagugugugugauauuu | 0.001 | 0.000 | 0.051 | 0.050 | 50.5 | 101 |
| hsa-miR-320a | aaaagcuggguugagagggcga | 0.059 | 0.143 | 0.539 | 0.224 | 9.1 | 102 |
| hsa-miR-320b | aaaagcuggguugagagggcaa | 0.028 | 0.064 | 0.260 | 0.195 | 9.2 | 62 |

TABLE 5

| miR (56 miR) having | | | | | | | |
|---|---|---|---|---|---|---|---|
| significant | | HC (n = 10) | | PC (n = 12) | | Fold Change | SEQ |
| difference (p < 0.05) | Sequence | mean (%) | SD | mean (%) | SD | (PC/HC) | ID NO: |
| hsa-miR-320c | aaaagcuggguugagagggu | 0.199 | 0.114 | 0.392 | 0.248 | 2.0 | 63 |
| hsa-miR-320d | aaaagcuggguugagagga | 0.655 | 0.291 | 1.123 | 0.580 | 1.7 | 64 |
| hsa-miR-324-3p | acugccccaggugcugcugg | 0.163 | 0.179 | 1.666 | 1.230 | 10.2 | 103 |
| hsa-miR-371a-5p | acucaaacugugggggcacu | 0.007 | 0.022 | 0.300 | 0.249 | 43.6 | 104 |
| hsa-miR-451a | aaaccguuaccauuacugaguu | 32.297 | 6.042 | 47.717 | 15.264 | 1.3 | 70 |
| hsa-miR-483-5p | aagacgggaggaaagaagggag | 0.308 | 0.216 | 0.816 | 0.533 | 2.6 | 71 |
| hsa-miR-486-5p | uccuguacugagcugccccgag | 0.373 | 0.266 | 0.678 | 0.471 | 1.8 | 72 |
| hsa-miR-493-5p | uuguacaugguaggcuuucauu | 0.001 | 0.000 | 0.042 | 0.055 | 42.1 | 105 |
| hsa-miR-494 | ugaaacauacacgggaaaccuc | 0.012 | 0.038 | 0.405 | 0.334 | 33.7 | 106 |
| hsa-miR-498 | uuucaagccagggggcguuuuuc | 0.001 | 0.000 | 0.186 | 0.184 | 186.2 | 107 |
| hsa-miR-513a-5p | uucacagggaggugucau | 0.035 | 0.076 | 0.381 | 0.299 | 10.8 | 73 |
| hsa-miR-518e-5p | cucuagagggaagcgcuuucug | 0.001 | 0.000 | 0.127 | 0.140 | 127.4 | 108 |
| hsa-miR-525-5p | cuccagagggaugcacuuucu | 0.001 | 0.000 | 0.102 | 0.114 | 101.5 | 109 |
| hsa-miR-526b-5p | cucuugagggaagcacuuucugu | 0.001 | 0.000 | 0.160 | 0.180 | 160.5 | 110 |
| hsa-miR-574-5p | ugagugugugugugugagugugu | 0.080 | 0.142 | 0.386 | 0.140 | 4.8 | 111 |
| hsa-miR-584-5p | uuaugguuugccugggacugag | 0.001 | 0.000 | 0.939 | 0.475 | 938.6 | 112 |
| hsa-miR-623 | aucccuugcaggggcuguugggu | 0.001 | 0.000 | 0.096 | 0.097 | 96.1 | 113 |
| hsa-miR-631 | agaccuggcccagaccucag | 0.001 | 0.000 | 0.319 | 0.201 | 318.8 | 114 |
| hsa-miR-652-3p | aauggcgccacuaggguugug | 0.001 | 0.000 | 0.716 | 0.792 | 715.8 | 115 |
| hsa-miR-662 | ucccacguuguggcccagcag | 0.001 | 0.000 | 0.164 | 0.185 | 164.0 | 116 |
| hsa-miR-671-5p | aggaagcccuggaggggcuggag | 0.644 | 0.271 | 3.481 | 1.549 | 5.4 | 78 |
| hsa-miR-765 | uggaggagaaggaaggugaug | 0.110 | 0.110 | 0.502 | 0.287 | 4.6 | 81 |
| hsa-miR-874 | cugcccuggcccgagggaccga | 0.001 | 0.000 | 0.100 | 0.119 | 100.1 | 82 |
| hsa-miR-885-3p | aggcagcgggguguaguggaua | 0.001 | 0.000 | 0.022 | 0.024 | 22.1 | 117 |
| hsa-miR-92b-5p | agggacgggacgcggugcagug | 0.001 | 0.000 | 0.017 | 0.025 | 16.8 | 118 |
| hsa-miR-934 | acaguagagggaggaaucgcag | 0.001 | 0.000 | 0.055 | 0.059 | 54.6 | 119 |
| hsa-miR-936 | acaguagagggaggaaucgcag | 0.001 | 0.000 | 0.056 | 0.074 | 56.3 | 120 |
| hsa-miR-939 | uggggagcugaggcucugggggug | 0.051 | 0.088 | 0.566 | 0.415 | 11.0 | 85 |

3. Measurement of Exosomal miRNA Using the Plasma of Pancreatic Cancer Patients (2)

Measurement similar to “2. Measurement of exosomal miRNA using the plasma of pancreatic cancer patients (1)” was performed using the plasma specimens (n=12) derived from other pancreatic cancer patients

More specifically, array data of the plasma specimens of 13 healthy controls and 12 pancreatic cancer patients before chemotherapy were calculated as a signal intensity. After correction with the plasma volume, thus calculated value was designated as a normalized signal intensity (AU). Test (Student's t-test) was performed for the healthy controls and a group of pancreatic cancer cases. The miRNA having a high mean value in the pancreatic cancer group and having a p value not more than 0.05 was determined as significantly high in comparison to healthy controls.

A profile of exosomal miRNA was prepared from the plasma by using a microarray and it was weighed against the analysis data of the plasma of healthy controls and colon cancer patients.

Concentration of the exosome fraction, profiling by a microarray, and subsequent data analysis were performed in manners similar to those described above in 1.

As a result, it has been found that nine miRNAs shown in Table 6, as well as the miRNAs detected in the above-mentioned “preparation of a profile of an exosome using the plasma of pancreatic cancer patients (1)” are detected at a significantly high level (p<0.05) compared with the healthy controls (n=13).

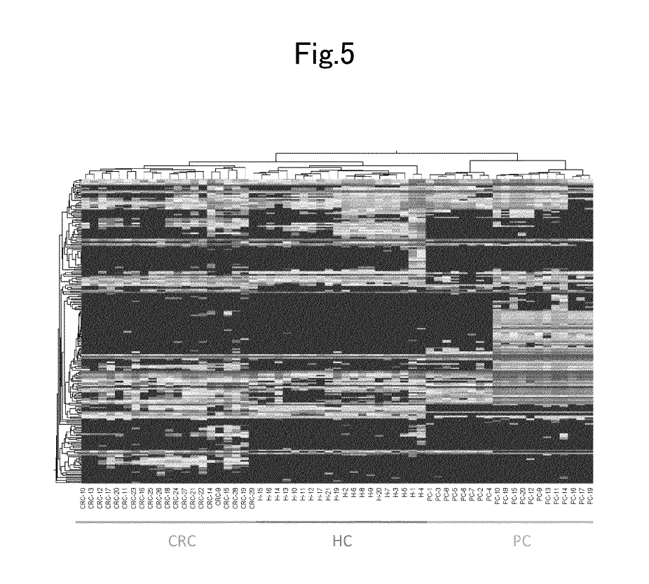

Further, as shown in FIG. 5, there is a large difference in the profile of the exosomal miRNA from the plasma between colon cancer patients and pancreatic cancer patients, strongly suggesting the possibility that the profile is specific to the type of cancer.

TABLE 6

| panc cancer cells | | | | |
|---|---|---|---|---|
| exosomes | | | | |
| | HC | | | |
| | normalized | PC | Student's t- | SEQ ID |
| 85miR | intensities | normalized | test P value | NO: |
| hsa-let-7a-5p | 13.6 | 30.3 | 0.0013 | 1 |
| hsa-let-7f-5p | 16 | 28.8 | 0.0181 | 5 |
| hsa-let-7i-5p | 5.8 | 12.2 | 0.0042 | 7 |
| hsa-miR-1268a | 15.1 | 61.6 | 0.0000 | 19 |
| hsa-miR-144-3p | 34.7 | 90.3 | 0.0087 | 26 |
| hsa-miR-15a-5p | 14 | 27 | 0.0174 | 30 |
| hsa-miR-16-5p | 89.6 | 156.5 | 0.0362 | 32 |
| hsa-miR-22-3p | 16.3 | 25.9 | 0.0499 | 42 |
| hsa-miR-720 | 69.5 | 96 | 0.0017 | 80 |

4. Identification of Colon Cancer Marker

Exosomal miRNA profiling was performed using the serum specimens (n=11) of healthy controls and serum specimens of colon cancer patients classified by TNM stage (Stages I (n=20), II (n=20), IIIa (n=20), IIIb (n=16), and IV (n=12)) and miRNAs specific to colon cancer were selected.

First, after dilution of a serum specimen (1 mL) with PBS to 10 times, an exosome fraction was concentrated by ultracentrifugation to prepare an RNA as a template of microarray analysis.

A profile of the exosomal miRNA from the serum was prepared using a microarray and analysis data of the serum of the colon cancer patients were weighed against those of the healthy controls.

Concentration of the exosome fraction, profiling using a microarray, and subsequent data analysis were performed in manners similar to those described above in 1.

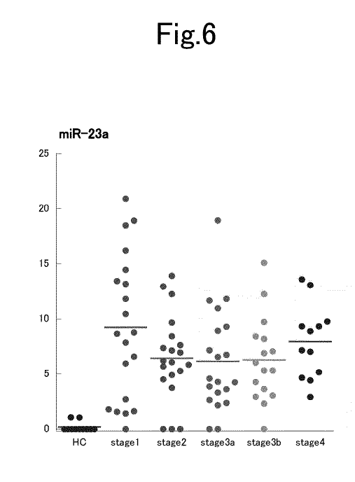

The results are shown in FIG. 6. As is apparent from this drawing, miR-23a-3p was detected at a significantly high level in any TNM stage compared with the healthy controls (n=11). In addition, almost no miR-23a-3p was detected from the serum of healthy controls.

5. Measurement of Concentration Before and after Operation

Paired serum specimens before and after cancer resection were collected from each of colon cancer patients (Stages I (n=6), II (n=5), Ma (n=5), Mb (n=5), IV (n=3)) and it was studied whether the miR-23a-3p decreases after the operation or not.

A profile of the exosomal miRNA from the serum was prepared using a microarray and analysis data of the serum of the colon cancer patients after operation were weighed against those of the patients before operation.

Concentration of the exosome fraction, profiling using a microarray, and subsequent data analysis were performed in manners similar to those described above in 1.

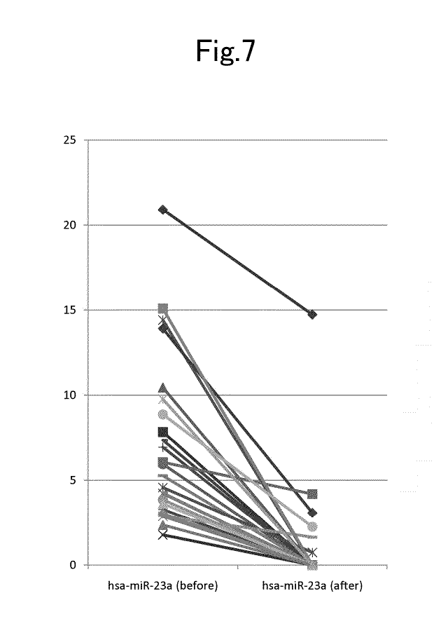

The results are shown in FIG. 7. As is apparent from this drawing, the miR-23a-3p is detected at a substantially low level after operation compared with that before operation. Further, the decrease after operation was observed in any TNM stage.

### Sequence Listing Free Text

SEQ ID NO: 1 to 120 show the sequence of miRNA.

1. A method of detecting pancreatic cancer of a subject, comprising measuring, in a sample derived from the subject, at least one miRNA selected from the following (i) and (ii):
(i) an miRNA having a sequence represented by any of SEQ ID NO: 1 to 120; and
(ii) an miRNA that hybridizes, under stringent conditions, with a nucleic acid having a sequence complementary to the miRNA (i) and having from 17 to 30 bases.

2. The detection method according to claim 1, wherein the miRNA selected from (i) has a sequence represented by any of SEQ ID NO: 1 to 3, 5, 7, 12, 13, 19 to 21, 26, 27, 30, 32, 35, 36, 42, 50, 62 to 64, 70 to 73, 78, 80, 81, 82, and 85 to 120.

3. The detection method according to claim 1 or 2, wherein the sample derived from a subject is prepared by a method comprising:
concentrating an exosome fraction of the plasma collected from the subject and;
extracting an miRNA from the exosome fraction.

4. A kit for carrying out the detection method as claimed in any one of claims 1 to 3, comprising a solid-phase support having, fixed thereto, a DNA that hybridizes with the miRNA selected from (i) and (ii).

5. A kit for carrying out the detection method as claimed in any one of claims 1 to 3, comprising a primer set necessary for amplifying the miRNA selected from (i) and (ii) or a nucleic acid complementary thereto by PCR assay.


SEQUENCE LISTING (120 sequences)

SEQ ID NO: 1 - RNA, 22 nt, Homo sapiens
  hsa-let-7a-5p
ugagguaguagguuguauaguu

SEQ ID NO: 2 - RNA, 22 nt, Homo sapiens
  hsa-let-7b-5p
ugagguaguagguugugugguu

SEQ ID NO: 3 - RNA, 22 nt, Homo sapiens
  hsa-let-7c
ugagguaguagguuguaugguu

SEQ ID NO: 4 - RNA, 22 nt, Homo sapiens
  hsa-let-7d-5p
agagguaguagguugcauaguu

SEQ ID NO: 5 - RNA, 22 nt, Homo sapiens
  hsa-let-7f-5p
ugagguaguagauuguauaguu

SEQ ID NO: 6 - RNA, 22 nt, Homo sapiens
  hsa-let-7g-5p
ugagguaguaguuuguacaguu

SEQ ID NO: 7 - RNA, 22 nt, Homo sapiens
  hsa-let-7i-5p
ugagguaguaguuugugcuguu

SEQ ID NO: 8 - RNA, 23 nt, Homo sapiens
  hsa-miR-103a-3p
agcagcauuguacagggcuauga

SEQ ID NO: 9 - RNA, 21 nt, Homo sapiens
  hsa-miR-106b-5p
uaaagugcugacagugcagau

SEQ ID NO: 10 - RNA, 23 nt, Homo sapiens
  hsa-miR-107
agcagcauuguacagggcuauca

SEQ ID NO: 11 - RNA, 21 nt, Homo sapiens
  hsa-miR-1181
ccgucgccgccacccgagccg

SEQ ID NO: 12 - RNA, 21 nt, Homo sapiens
  hsa-miR-1207-5p
uggcagggaggcugggagggg

SEQ ID NO: 13 - RNA, 19 nt, Homo sapiens
  hsa-miR-1224-5p
gugaggacucgggaggugg

SEQ ID NO: 14 - RNA, 19 nt, Homo sapiens
  hsa-miR-1246
aauggauuuuuggagcagg

SEQ ID NO: 15 - RNA, 22 nt, Homo sapiens
  hsa-miR-125a-3p
acaggugagguucuugggagcc

SEQ ID NO: 16 - RNA, 22 nt, Homo sapiens
  hsa-miR-125b-2-3p
ucacaagucaggcucuugggac

SEQ ID NO: 17 - RNA, 22 nt, Homo sapiens
  hsa-miR-126-3p
ucguaccgugaguaauaaugcg

SEQ ID NO: 18 - RNA, 18 nt, Homo sapiens
  hsa-miR-1260a
aucccaccucugccacca

SEQ ID NO: 19 - RNA, 18 nt, Homo sapiens
  hsa-miR-1268a
cgggcguggugguggggg

SEQ ID NO: 20 - RNA, 17 nt, Homo sapiens
  hsa-miR-1275
gugggggagaggcuguc

SEQ ID NO: 21 - RNA, 19 nt, Homo sapiens
  hsa-miR-1290
uggauuuuuggaucaggga

SEQ ID NO: 22 - RNA, 22 nt, Homo sapiens
  hsa-miR-1305
uuuucaacucuaaugggagaga

SEQ ID NO: 23 - RNA, 22 nt, Homo sapiens
  hsa-miR-134
ugugacugguugaccagagggg

SEQ ID NO: 24 - RNA, 22 nt, Homo sapiens
  hsa-miR-135a-3p
uauagggauuggagccguggcg

SEQ ID NO: 25 - RNA, 23 nt, Homo sapiens
  hsa-miR-142-3p
uguaguguuuccuacuuuaugga

SEQ ID NO: 26 - RNA, 20 nt, Homo sapiens
  hsa-miR-144-3p
uacaguauagaugauguacu

SEQ ID NO: 27 - RNA, 22 nt, Homo sapiens
  hsa-miR-1471
gcccgcguguggagccaggugu

SEQ ID NO: 28 - RNA, 22 nt, Homo sapiens
  hsa-miR-150-5p
ucucccaacccuuguaccagug

SEQ ID NO: 29 - RNA, 21 nt, Homo sapiens
  hsa-miR-151a-5p
ucgaggagcucacagucuagu

SEQ ID NO: 30 - RNA, 22 nt, Homo sapiens
  hsa-miR-15a-5p
uagcagcacauaaugguuugug

SEQ ID NO: 31 - RNA, 22 nt, Homo sapiens
  hsa-miR-15b-5p
uagcagcacaucaugguuuaca

SEQ ID NO: 32 - RNA, 22 nt, Homo sapiens
  hsa-miR-16-5p
uagcagcacguaaauauuggcg

SEQ ID NO: 33 - RNA, 23 nt, Homo sapiens
  hsa-miR-17-5p
caaagugcuuacagugcagguag

SEQ ID NO: 34 - RNA, 23 nt, Homo sapiens
  hsa-miR-181a-5p
aacauucaacgcugucggugagu

SEQ ID NO: 35 - RNA, 22 nt, Homo sapiens
  hsa-miR-1914-3p
ggaggggucccgcacugggagg

SEQ ID NO: 36 - RNA, 22 nt, Homo sapiens
  hsa-miR-193b-5p
cgggguuuugagggcgagauga

SEQ ID NO: 37 - RNA, 23 nt, Homo sapiens
  hsa-miR-19a-3p
ugugcaaaucuaugcaaaacuga

SEQ ID NO: 38 - RNA, 23 nt, Homo sapiens
  hsa-miR-19b-3p
ugugcaaauccaugcaaaacuga

SEQ ID NO: 39 - RNA, 23 nt, Homo sapiens
  hsa-miR-20a-5p
uaaagugcuuauagugcagguag

SEQ ID NO: 40 - RNA, 23 nt, Homo sapiens
  hsa-miR-20b-5p
caaagugcucauagugcagguag

SEQ ID NO: 41 - RNA, 22 nt, Homo sapiens
  hsa-miR-21-5p
uagcuuaucagacugauguuga

SEQ ID NO: 42 - RNA, 22 nt, Homo sapiens
  hsa-miR-22-3p
aagcugccaguugaagaacugu

SEQ ID NO: 43 - RNA, 23 nt, Homo sapiens
  hsa-miR-221-3p
agcuacauugucugcuggguuuc

SEQ ID NO: 44 - RNA, 21 nt, Homo sapiens
  hsa-miR-222-3p
agcuacaucuggcuacugggu

SEQ ID NO: 45 - RNA, 22 nt, Homo sapiens
  hsa-miR-223-3p
ugucaguuugucaaauacccca

SEQ ID NO: 46 - RNA, 21 nt, Homo sapiens
  hsa-miR-224-5p
caagucacuagugguuccguu

SEQ ID NO: 47 - RNA, 21 nt, Homo sapiens
  hsa-miR-23a-3p
aucacauugccagggauuucc

SEQ ID NO: 48 - RNA, 21 nt, Homo sapiens
  hsa-miR-23b-3p
aucacauugccagggauuacc

SEQ ID NO: 49 - RNA, 22 nt, Homo sapiens
  hsa-miR-24-3p
uggcucaguucagcaggaacag

SEQ ID NO: 50 - RNA, 22 nt, Homo sapiens
  hsa-miR-25-3p
cauugcacuugucucggucuga

SEQ ID NO: 51 - RNA, 22 nt, Homo sapiens
  hsa-miR-26a-5p
uucaaguaauccaggauaggcu

SEQ ID NO: 52 - RNA, 21 nt, Homo sapiens
  hsa-miR-26b-5p
uucaaguaauucaggauaggu

SEQ ID NO: 53 - RNA, 21 nt, Homo sapiens
  hsa-miR-27a-3p
uucacaguggcuaaguuccgc

SEQ ID NO: 54 - RNA, 21 nt, Homo sapiens
  hsa-miR-27b-3p
uucacaguggcuaaguucugc

SEQ ID NO: 55 - RNA, 22 nt, Homo sapiens
  hsa-miR-29a-3p
uagcaccaucugaaaucgguua

SEQ ID NO: 56 - RNA, 22 nt, Homo sapiens
  hsa-miR-29c-3p
uagcaccauuugaaaucgguua

SEQ ID NO: 57 - RNA, 22 nt, Homo sapiens
  hsa-miR-30a-5p
uguaaacauccucgacuggaag

SEQ ID NO: 58 - RNA, 22 nt, Homo sapiens
  hsa-miR-30b-5p
uguaaacauccuacacucagcu

SEQ ID NO: 59 - RNA, 23 nt, Homo sapiens
  hsa-miR-30c-5p
uguaaacauccuacacucucagc

SEQ ID NO: 60 - RNA, 22 nt, Homo sapiens
  hsa-miR-30d-5p
uguaaacauccccgacuggaag

SEQ ID NO: 61 - RNA, 22 nt, Homo sapiens
  hsa-miR-30e-5p
uguaaacauccuugacuggaag

SEQ ID NO: 62 - RNA, 22 nt, Homo sapiens
  hsa-miR-320b
aaaagcuggguugagagggcaa

SEQ ID NO: 63 - RNA, 20 nt, Homo sapiens
  hsa-miR-320c
aaaagcuggguugagagggu

SEQ ID NO: 64 - RNA, 19 nt, Homo sapiens
  hsa-miR-320d
aaaagcuggguugagagga

SEQ ID NO: 65 - RNA, 21 nt, Homo sapiens
  hsa-miR-331-3p
gccccugggccuauccuagaa

SEQ ID NO: 66 - RNA, 22 nt, Homo sapiens
  hsa-miR-345-5p
gcugacuccuaguccagggcuc

SEQ ID NO: 67 - RNA, 22 nt, Homo sapiens
  hsa-miR-365a-3p
uaaugccccuaaaaauccuuau

SEQ ID NO: 68 - RNA, 23 nt, Homo sapiens
  hsa-miR-423-5p
ugaggggcagagagcgagacuuu

SEQ ID NO: 69 - RNA, 23 nt, Homo sapiens
  hsa-miR-425-5p
aaugacacgaucacucccguuga

SEQ ID NO: 70 - RNA, 22 nt, Homo sapiens
  hsa-miR-451a
aaaccguuaccauuacugaguu

SEQ ID NO: 71 - RNA, 22 nt, Homo sapiens
  hsa-miR-483-5p
aagacgggaggaaagaagggag

SEQ ID NO: 72 - RNA, 22 nt, Homo sapiens
  hsa-miR-486-5p
uccuguacugagcugccccgag

SEQ ID NO: 73 - RNA, 18 nt, Homo sapiens
  hsa-miR-513a-5p
uucacagggaggugucau

SEQ ID NO: 74 - RNA, 22 nt, Homo sapiens
  hsa-miR-601
uggucuaggauuguuggaggag

SEQ ID NO: 75 - RNA, 21 nt, Homo sapiens
  hsa-miR-610
ugagcuaaaugugugcuggga

SEQ ID NO: 76 - RNA, 22 nt, Homo sapiens
  hsa-miR-630
aguauucuguaccagggaaggu

SEQ ID NO: 77 - RNA, 22 nt, Homo sapiens
  hsa-miR-654-5p
uggugggccgcagaacaugugc

SEQ ID NO: 78 - RNA, 23 nt, Homo sapiens
  hsa-miR-671-5p
aggaagcccuggaggggcuggag

SEQ ID NO: 79 - RNA, 23 nt, Homo sapiens
  hsa-miR-708-5p
aaggagcuuacaaucuagcuggg

SEQ ID NO: 80 - RNA, 17 nt, Homo sapiens
  hsa-miR-720
ucucgcuggggccucca

SEQ ID NO: 81 - RNA, 21 nt, Homo sapiens
  hsa-miR-765
uggaggagaaggaaggugaug

SEQ ID NO: 82 - RNA, 22 nt, Homo sapiens
  hsa-miR-874
cugcccuggcccgagggaccga

SEQ ID NO: 83 - RNA, 22 nt, Homo sapiens
  hsa-miR-92a-3p
uauugcacuugucccggccugu

SEQ ID NO: 84 - RNA, 23 nt, Homo sapiens
  hsa-miR-93-5p
caaagugcuguucgugcagguag

SEQ ID NO: 85 - RNA, 24 nt, Homo sapiens
  hsa-miR-939
uggggagcugaggcucugggggug

SEQ ID NO: 86 - RNA, 23 nt, Homo sapiens
  hsa-miR-1182
gagggucuugggagggaugugac

SEQ ID NO: 87 - RNA, 27 nt, Homo sapiens
  hsa-miR-1183
cacuguaggugauggugagagugggca

SEQ ID NO: 88 - RNA, 20 nt, Homo sapiens
  hsa-miR-1208
ucacuguucagacaggcgga

SEQ ID NO: 89 - RNA, 22 nt, Homo sapiens
  hsa-miR-122-5p
uggagugugacaaugguguuug

SEQ ID NO: 90 - RNA, 26 nt, Homo sapiens
  hsa-miR-1226-5p
gugagggcaugcaggccuggaugggg

SEQ ID NO: 91 - RNA, 20 nt, Homo sapiens
  hsa-miR-124-3p
uaaggcacgcggugaaugcc

SEQ ID NO: 92 - RNA, 22 nt, Homo sapiens
  hsa-miR-1287
ugcuggaucagugguucgaguc

SEQ ID NO: 93 - RNA, 22 nt, Homo sapiens
  hsa-miR-141-3p
uaacacugucugguaaagaugg

SEQ ID NO: 94 - RNA, 22 nt, Homo sapiens
  hsa-miR-144-5p
ggauaucaucauauacuguaag

SEQ ID NO: 95 - RNA, 22 nt, Homo sapiens
  hsa-miR-150-3p
cugguacaggccugggggacag

SEQ ID NO: 96 - RNA, 22 nt, Homo sapiens
  hsa-miR-181c-3p
aaccaucgaccguugaguggac

SEQ ID NO: 97 - RNA, 22 nt, Homo sapiens
  hsa-miR-184
uggacggagaacugauaagggu

SEQ ID NO: 98 - RNA, 22 nt, Homo sapiens
  hsa-miR-193a-5p
ugggucuuugcgggcgagauga

SEQ ID NO: 99 - RNA, 20 nt, Homo sapiens
  hsa-miR-202-3p
agagguauagggcaugggaa

SEQ ID NO: 100 - RNA, 22 nt, Homo sapiens
  hsa-miR-22-5p
aguucuucaguggcaagcuuua

SEQ ID NO: 101 - RNA, 22 nt, Homo sapiens
  hsa-miR-32-3p
caauuuagugugugugauauuu

SEQ ID NO: 102 - RNA, 22 nt, Homo sapiens
  hsa-miR-320a
aaaagcuggguugagagggcga

SEQ ID NO: 103 - RNA, 20 nt, Homo sapiens
  hsa-miR-324-3p
acugccccaggugcugcugg

SEQ ID NO: 104 - RNA, 20 nt, Homo sapiens
  hsa-miR-371a-5p
acucaaacugugggggcacu

SEQ ID NO: 105 - RNA, 22 nt, Homo sapiens
  hsa-miR-493-5p
uuguacaugguaggcuuucauu

SEQ ID NO: 106 - RNA, 22 nt, Homo sapiens
  hsa-miR-494
ugaaacauacacgggaaaccuc

SEQ ID NO: 107 - RNA, 23 nt, Homo sapiens
  hsa-miR-498
uuucaagccagggggcguuuuuc

SEQ ID NO: 108 - RNA, 22 nt, Homo sapiens
  hsa-miR-518e-5p
cucuagagggaagcgcuuucug

SEQ ID NO: 109 - RNA, 21 nt, Homo sapiens
  hsa-miR-525-5p
cuccagagggaugcacuuucu

SEQ ID NO: 110 - RNA, 23 nt, Homo sapiens
  hsa-miR-526b-5p
cucuugagggaagcacuuucugu

SEQ ID NO: 111 - RNA, 23 nt, Homo sapiens
  hsa-miR-574-5p
ugagugugugugugugagugugu

SEQ ID NO: 112 - RNA, 22 nt, Homo sapiens
  hsa-miR-584-5p
uuaugguuugccugggacugag

SEQ ID NO: 113 - RNA, 23 nt, Homo sapiens
  hsa-miR-623
aucccuugcaggggcuguugggu

SEQ ID NO: 114 - RNA, 20 nt, Homo sapiens
  hsa-miR-631
agaccuggcccagaccucag

SEQ ID NO: 115 - RNA, 21 nt, Homo sapiens
  hsa-miR-652-3p
aauggcgccacuaggguugug

SEQ ID NO: 116 - RNA, 21 nt, Homo sapiens
  hsa-miR-662
ucccacguuguggcccagcag

SEQ ID NO: 117 - RNA, 22 nt, Homo sapiens
  hsa-miR-885-3p
aggcagcgggguguaguggaua

SEQ ID NO: 118 - RNA, 22 nt, Homo sapiens
  hsa-miR-92b-5p
agggacgggacgcggugcagug

SEQ ID NO: 119 - RNA, 22 nt, Homo sapiens
  hsa-miR-934
ugucuacuacuggagacacugg

SEQ ID NO: 120 - RNA, 22 nt, Homo sapiens
  hsa-miR-936
acaguagagggaggaaucgcag